(19) United States
(12) Patent Application Publication
(10) Pub. No.: US 2021/0059827 A1
(43) Pub. Date: Mar. 4, 2021

(54) Orthopaedic Surgical Instrument And Method For Implanting a Mobile Bearing Knee Prosthesis

(71) Applicant: DEPUY IRELAND UNLIMITED COMPANY, Cork (IE)

(72) Inventors: THIAGO LOPES AMARAL, COLUMBIA CITY, IN (US); LAUREN A. FERRIS, ELKHART, IN (US); LINDSAY L. GILSON, COLUMBIA CITY, IN (US); TRENT M. GLASSLEY, FORT WAYNE, IN (US); COLIN M. LANK, FORT WAYNE, IN (US); GARY M. LINDSAY, FORT WAYNE, IN (US); JEREMIAH M. LEWIS, LEESBURG, IN (US); AARON J. MATYAS, FORT WAYNE, IN (US); STEPHANIE A. RECKER, FORT WAYNE, IN (US); NATHAN C. REEDER, WARSAW, IN (US); DAVID E. ROTTGER, AUBURN, IN (US); PAUL B. SADE, CHURUBUSCO, IN (US); BARRY A. SCHNIEDERS, PLYMOUTH, IN (US)

(21) Appl. No.: 17/005,538

(22) Filed: Aug. 28, 2020

Related U.S. Application Data
(60) Provisional application No. 62/892,964, filed on Aug. 28, 2019.

(51) Int. Cl. A61F 2/38; A61F 2/30; A61F 2/46

(52) U.S. Cl. CPC A61F 2/3859; A61F 2/389; A61F 2002/30881; A61F 2/461; A61F 2002/3863; A61F 2/30771

(57) ABSTRACT

An orthopaedic surgical instrument configured to assist a surgeon seat or engage a femoral component with a tibial bearing or other tibial component. The instrument may include a displacement device configured to displace the tibia and femur to permit the surgeon to position a femoral component for seating on a tibial bearing and/or a retaining mechanism configured to maintain the tibial bearing in rotational alignment with the femoral component while seating the femoral component on the tibial bearing.

This application claims priority under 35 U.S.C. § 119 to U.S. Provisional Application No. 62/892,964, which is expressly incorporated herein by reference.

### TECHNICAL FIELD

The present disclosure relates generally to orthopaedic surgical instruments and methods for implanting orthopaedic prostheses, and, more particularly, to orthopaedic surgical instruments and methods for implanting prostheses in knee replacement surgeries.

### BACKGROUND

Joint arthroplasty is a well-known surgical procedure by which a diseased and/or damaged natural joint is replaced by a prosthetic joint. A typical knee prosthesis includes a tibial component and a femoral component adapted to contact a bearing surface of the tibial component. The tibial component typically includes a stem extending distally therefrom that is implanted in a prepared medullary canal of the patient's tibia.

Some tibial components are assemblies formed from multiple components. For example, one common tibial component assembly includes a tibial tray configured to be implanted in a patient's tibia and a tibial insert or bearing configured to be positioned between the tibial tray and the femoral component. The tibial bearing is configured to engage the femoral component such that the femoral component articulates on the tibial bearing as the knee joint is moved between extension and flexion. The tibial bearing may be in a fixed position relative to the tibial tray or it may be configured to rotate or pivot relative to the tibial tray. Such moveable or rotatable tibial bearings are commonly referred to as “mobile bearings.” An exemplary mobile bearing design is shown and described in U.S. Pat. No. 6,443,991, which is expressly incorporated herein by reference.

To facilitate the replacement of the natural joint with the knee prosthesis, orthopaedic surgeons use a variety of orthopaedic surgical instruments such as, for example, trial components, drill guides, reamers, impactors, and other surgical instruments.

### INTRODUCTION

According to one aspect of the disclosure, an orthopaedic surgical instrument is disclosed. The orthopaedic surgical instrument includes a displacement device configured to displace the tibia and/or femur to permit the surgeon to position a femoral component for seating on a tibial bearing. The instrument also includes a retaining mechanism configured to maintain the tibial bearing in rotational alignment with the femoral component while seating the femoral component on the tibial bearing.

In some embodiments, the displacement device may be omitted. In other embodiments, the retaining mechanism may be omitted from the orthopaedic surgical instrument.

An orthopaedic prosthesis system including the femoral component, the tibial bearing, and the orthopaedic surgical instrument is also disclosed.

A method of implanting an orthopaedic prosthesis is also disclosed.

### BRIEF DESCRIPTION OF THE DRAWINGS

The detailed description particularly refers to the following figures, in which:

FIG. 1 is an exploded view of an orthopaedic prosthesis system;

FIG. 2 is a cross-sectional elevation view of a femoral component of the orthopaedic prosthesis system taken along the line 2-2 in FIG. 2;

FIG. 3 illustrates a portion of a surgical procedure for implanting the components of the orthopaedic prosthesis system of FIG. 1;

FIG. 4 illustrates another portion of the surgical procedure for implanting the components of the orthopaedic prosthesis system;

FIG. 5 is a perspective view of an orthopaedic surgical instrument for use with the components of the orthopaedic prosthesis system of FIG. 1;

FIG. 6 is an elevation view of the orthopaedic surgical instrument of FIG. 5 used during the surgical procedure with the components of the orthopaedic prosthesis system of FIG. 1;

FIG. 7 is a perspective view of another embodiment of an orthopaedic surgical instrument for use with the components of the orthopaedic prosthesis system of FIG. 1;

FIG. 8 is an elevation view of the orthopaedic surgical instrument of FIG. 7 used during a surgical procedure with the components of the orthopaedic prosthesis system of FIG. 1;

FIG. 9 is an elevation view of another embodiment of an orthopaedic surgical instrument used during a surgical procedure with the components of the orthopaedic prosthesis system of FIG. 1;

FIG. 10 is an elevation view of another embodiment of an orthopaedic surgical instrument used during a surgical procedure with the components of the orthopaedic prosthesis system of FIG. 1;

FIG. 11 is a perspective view of another embodiment of an orthopaedic surgical instrument for use with the components of the orthopaedic prosthesis system of FIG. 1;

FIG. 12 is an elevation view of another embodiment of an orthopaedic surgical instrument used during a surgical procedure with the components of the orthopaedic prosthesis system of FIG. 1;

FIG. 13A is a perspective view of another embodiment of an orthopaedic surgical instrument;

FIG. 13B is a top plan view of the instrument of FIG. 13A;

FIG. 13C is an elevation view of the instrument of FIG. 13A used during a surgical procedure with the components of the orthopaedic prosthesis system of FIG. 1;

FIG. 14A is an elevation view of another embodiment of an orthopaedic surgical instrument;

FIG. 14B is a top plan view of the instrument of FIG. 14A;

FIG. 14C is an elevation view of the instrument of FIG. 14A used during a surgical procedure with the components of the orthopaedic prosthesis system of FIG. 1;

FIG. 15A is a top plan view of another embodiment of an orthopaedic surgical instrument;

FIG. 15B is an elevation view of the instrument of FIG. 15A used during a surgical procedure with the components of the orthopaedic prosthesis system of FIG. 1;

FIG. 16 is an elevation view of the instrument of FIG. 15A used with another instrument during a surgical procedure with the components of the orthopaedic prosthesis system of FIG. 1;

FIGS. 17A and B are elevation views of other embodiments of the orthopaedic surgical instruments;

FIG. 18A is a front elevation view of the implants of the orthopaedic prosthesis system of FIG. 1;

FIG. 18B is an elevation view of another embodiment of an orthopaedic surgical instrument used during a surgical procedure with the components of the orthopaedic prosthesis system of FIG. 1;

FIG. 19A is an elevation view of another embodiment of an orthopaedic surgical instrument;

FIG. 19B is a top plan view of the instrument of FIG. 13A; and

FIG. 19C is an elevation view of the instrument of FIG. 13A used during a surgical procedure with the components of the orthopaedic prosthesis system of FIG. 1.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and orthopaedic surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

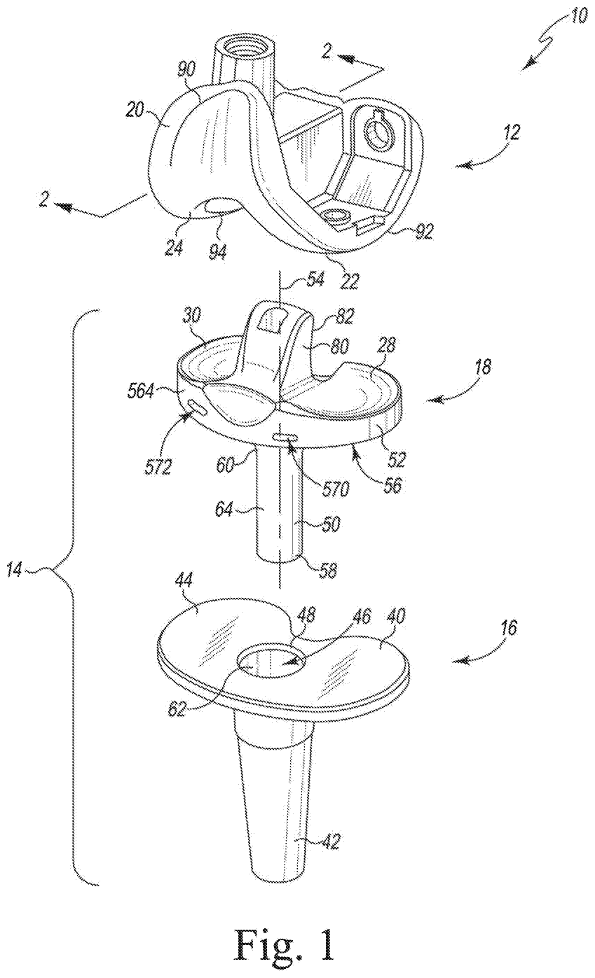

Referring now to FIG. 1, an orthopaedic prosthesis system 10 comprises a femoral component 12 and a tibial component 14. The tibial component 14 includes a tibial tray 16 and a bearing 18, which rotatably mounts on the tibial tray 16. The femoral component 12 includes a number of convex articulation surfaces 20, including condyle surfaces 22, 24, which are configured to engage corresponding concave articulation surfaces, including bearing surfaces 28, 30, of the bearing 18. As described in greater detail below, the femoral component 12 is configured to seat on the bearing 18, with its condyle surfaces 22, 24 engaging bearing surfaces 28, 30, respectively, of the bearing 18.

The tibial tray 16 includes a platform 40 and a tibial stem 42 that extends downwardly or inferiorly from the platform 40. The platform 40 includes a substantially planar proximal surface 44 and a socket 46 that extends downwardly from an opening 48 in the platform. The socket 46 is sized to receive a stem 50 of the bearing 18.

The bearing 18 includes a body 52 and the stem 50 extending inferiorly from the body 52. The body 52 includes the medial and lateral bearing surfaces 28, 30, which, as described above, are shaped to engage the femoral component 12. As shown in FIG. 1, the stem 50 extends along a longitudinal axis 54 from a distal surface 56 of the body 52, which is shaped to engage the proximal surface 44 of the tibial tray 16, to a distal tip 58. The stem 50 of the bearing 18 is provided with a generally tapered portion 60, which rotatably seats in a generally proximal tapered portion 62 of the socket 46. The stem 50 terminates in a distal cylindrical portion 64, which seats in a mating distal portion (not shown) of the socket 46. In the illustrative embodiment, the shape of the stem and socket permits relatively free rotational movement of the bearing 18 relative to the tibia tray 16 about the axis 54. However, it will be understood that the stem and socket need not be provided with such cylindrical and tapered portions. Other mounting configurations which allow for relatively free rotational movement may be included.

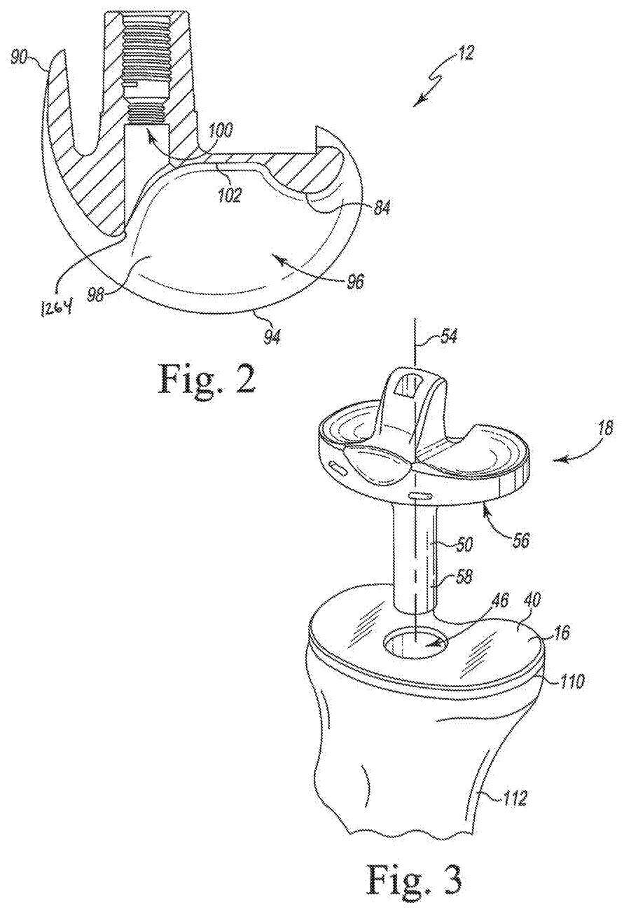

In the illustrative embodiment, the bearing 18 also includes a spine 80, which is sometimes referred to as an eminence or post. The spine 80 is located between the bearing surfaces 28, 30. The spine 80 includes a posterior surface 82 that is configured to engage a posterior cam 84 (see FIG. 2) of the femoral component 12 over a range of flexion. It should be appreciated that in other embodiments the spine may be omitted. As shown in FIG. 1, the bearing 18 is formed as a single, monolithic component from a plastic material such as ultra-high molecular weight polyethylene (UHMWPE). The tibial tray and femoral component are illustratively formed from a suitable implant-grade metallic material. In some embodiments, the tibial tray and/or femoral component may be formed from a plastic, ceramic, or composite material including a combination of plastic, ceramic, or metallic materials.

The femoral component 12 includes an anterior flange 90 that transitions to a pair of condyles 92, 94. A notch 96 is defined between the condyles 92, 94 by a number of inner walls 98. As shown in FIG. 2, the cam 84 is positioned at the posterior end of the notch 96. The femoral component 12 illustratively has a threaded aperture 100 extending through an inner wall 102 positioned at the base of the notch 96.

Referring now FIG. 3, a surgeon may prepare a patient's bones to receive the implant components of the orthopaedic prosthesis system 10. The surgeon may use alignment guides to identify cutting planes and a cutting block (not shown) to resect a proximal end 110 of a patient's tibia 112. The surgeon may also utilize one or more drills, reamers, and guide towers to prepare a cavity in the proximal end 110 of the patient's tibia 112 sized to receive the stem 42 of the tibial tray 16. To prepare the distal end 114 of the patient's femur 116, the surgeon may use additional surgical tools, including alignment guides, cutting blocks, and trials, to resect portions of the distal end of the femur.

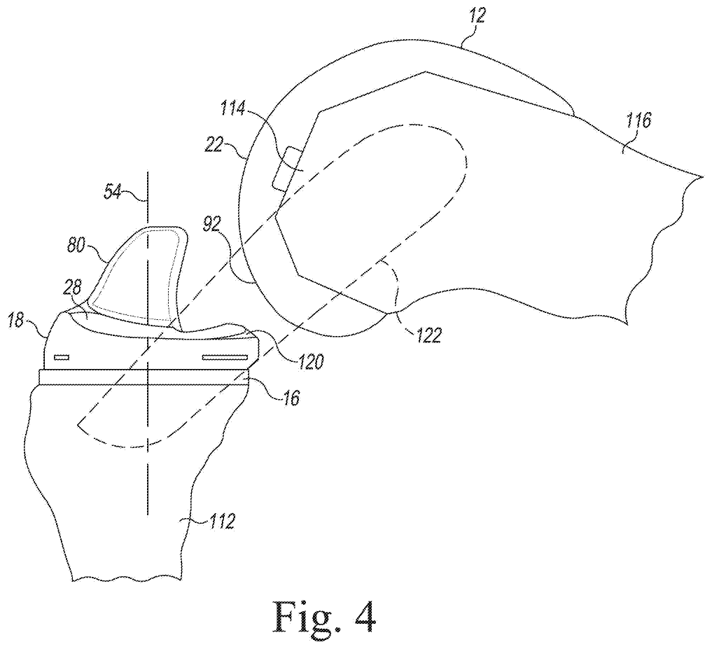

The surgeon may implant the tibial tray 16, as shown in FIG. 3. To mount the tibial bearing 18 on the tibial tray 16, the surgeon may align the distal tip 58 of the bearing stem 50 with the socket 46 of the tibial tray 16, as shown in FIG. 3. The surgeon may advance the tibial bearing 18 distally, lowering the stem 50 into the socket 46 and positioning the distal surface 56 of the bearing 18 in contact with the proximal surface 44 of the tibial tray 16, as shown in FIG. 4. The surgeon may attach the femoral component 12 to the surgical-prepared distal end 114, as shown in FIG. 4.

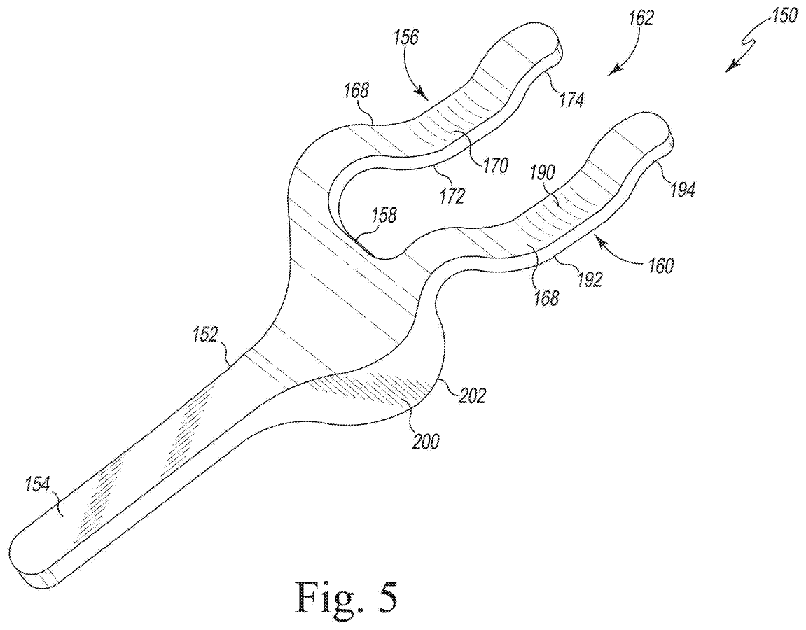

The patient's medial and lateral collateral ligaments (including the lateral ligament 122 shown in FIG. 4) resist displacement of the femur 116 and the tibia 112. To engage the femoral component 12 with the tibial bearing 18, the surgeon may counter the tension of the collateral ligaments and advance the condyles 22, 24 over the posterior edge 120 of the tibial bearing to seat the femoral component 12 on the tibial bearing. To prevent rotation of the tibial bearing 18 relative to the tibial tray 16, the surgeon may use, for example, the alignment tool 150 of FIG. 5 to hold the tibial bearing in position. The alignment tool 150 is also configured to displace the femur and the tibia to move the femoral component into position on the tibial bearing, as described in greater detail below.

The alignment tool 150 of the system 10 includes an elongated body 152 having a handle 154 sized to be gripped by a surgeon or other user. A medial prong 156 extends from an end 158 of the elongated body 152. The alignment tool 150 includes a lateral prong 160 also extending from the end 158 that is spaced apart from the medial prong 156. A channel 162 sized to receive the spine 80 is defined between the prongs 156, 160. In the illustrative embodiment, the tool 150 is formed as a single, monolithic component from a plastic such as polyethylene. In other embodiments, it may be formed as separate components, which may be later assembled into a single device. Some or all of the tool 150, like the other tools described in this specification, may be formed from a metallic material such as, for example, stainless steel or a combination of metal and plastic materials.

The medial prong 156 includes an arm 168 that has a concave proximal surface 170 shaped to engage the convex condyle surface 20 of the femoral component 12. The medial prong 156 also includes a convex distal surface 172 that is positioned opposite the proximal surface 170. In the illustrative embodiment, the convex distal surface 172 is configured to engage the concave bearing surface 26 of the tibial bearing 18. The medial prong 156 also includes an outer flange 174, which extends away from the arm 168.

The lateral prong 160 includes an arm 188 that has a concave proximal surface 190 shaped to engage the convex condyle surface 22 of the femoral component 12. The lateral prong 160 also includes a convex distal surface 192 that is positioned opposite the proximal surface 190. In the illustrative embodiment, the convex distal surface 192 is configured to engage the concave bearing surface 28 of the tibial bearing 18. The lateral prong 160 also includes an outer flange 194, which extends away from the arm 188.

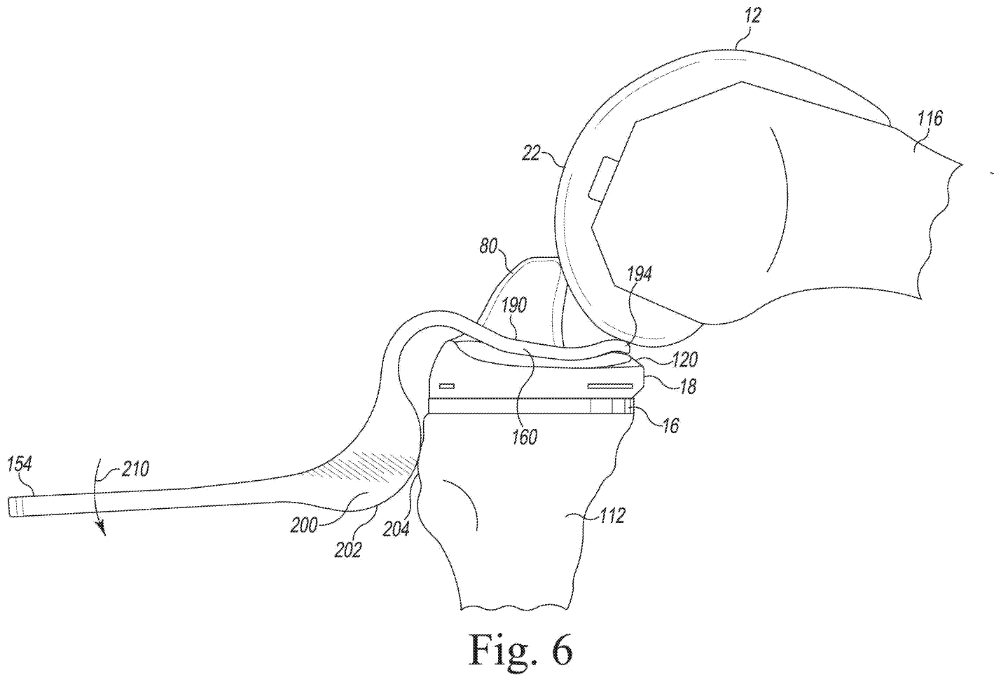

As shown in FIG. 5, the alignment tool 150 also includes a tab 200 extending from the elongated body 152. The tab 200 includes a convex outer surface 202 that is shaped to engage an anterior surface 204 of the patient's tibia 112 (see FIG. 6). In the illustrative embodiment, the tab 200 is shaped to act as a pivot against the patient's tibia, while the prongs 156, 160 displace the femur and tibia to position the femoral component 12 for seating on the tibial bearing 18. The prongs 156, 160 are also shaped to engage the spine 80 to prevent or inhibit the rotation of the tibial bearing about the axis 54 relative to the tibial tray 16 to keep the spine 80 aligned for positioning within the intercondylar notch 96 of the femoral component 12.

In use, the surgeon may place the femur in deep flexion, as shown in FIG. 6, with the tibial bearing 18 mounted on the tibial tray 16. The surgeon may align the channel 162 of the tool 150 with the spine 80 of the bearing 18 before advancing the prongs 160, 156 over the bearing surfaces 28, 30. As the prongs 160, 156 move over the bearing surfaces 28, 30, the spine 80 is received in the channel 162 and the tips of the outer flanges 194, 174 are positioned in the gap between the femoral component 12 and the posterior edge 120 of the tibial bearing 18. To displace the femur and the tibia, the surgeon may rotate the handle 154 of the tool 150 downward (as indicated by arrow 210) to position the tab 200 in contact with the anterior surface 204 of the tibia.

The surgeon may continue pushing down on the handle to raise the femur (or lower the tibia), while drawing the femur and the tibia closer together (by a combination of pushing backward with the tab 200 on the tibia and drawing the femur forward along the prongs 156, 160), thereby positioning the femoral component 12 for seating on the tibial bearing 18. With the spine 80 positioned between the prongs 156, 160, rotation of the tibial bearing 18 is inhibited such that the bearing surfaces 28, 30 are aligned with the condyles 92, 94, respectively, of the femoral component 12 and the spine 80 remains aligned with the notch 96. When the condyles 94, 92 are engaged with the concave proximal surfaces 170, 190 of the tool 150 over the bearing surfaces 30, 28, the surgeon may withdraw the prongs 156, 160 from between the femoral component 12 and the tibial bearing 18, thereby allowing the femoral component 12 to seat on the bearing 18.

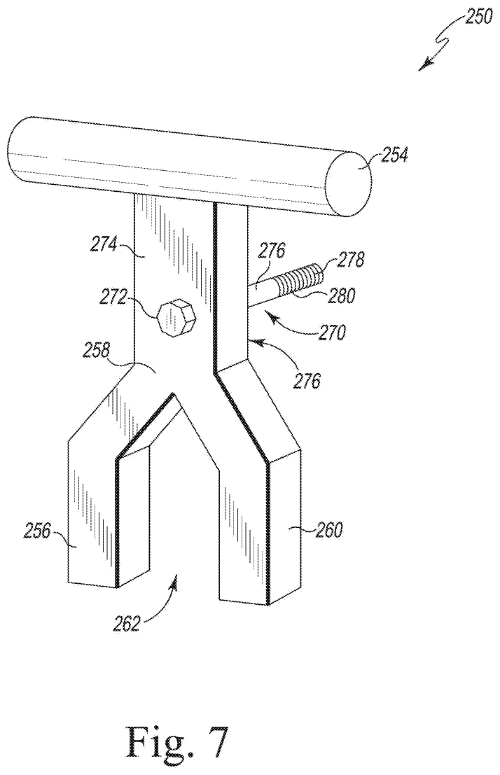

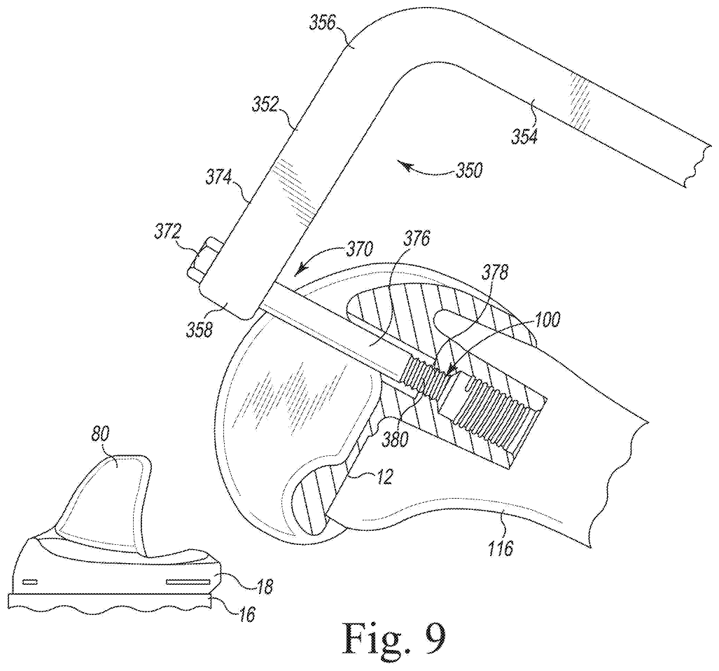

Referring now to FIG. 7, another embodiment of an alignment tool (hereinafter alignment tool 250) is shown. Like the alignment tool 150, the tool 250 is configured to hold the tibial bearing in position and displace the femur and the tibia such that the bones are moved to position the femoral component relative to the tibial bearing. The alignment tool 250 includes an elongated body 252 and a handle 254 positioned at one end of the body 252, which is sized to be gripped by a surgeon or other user. In the illustrative embodiment, the handle 254 is T-shaped, but it should be appreciated that in other embodiments it may take other forms, including, for example, the shape of the handle shown in FIG. 9. A medial prong 256 extends from a distal end 258 of the elongated body 252, and the alignment tool 250 includes a lateral prong 260 also extending from the end 258. The lateral prong 260 is spaced apart from the medial prong 256 such that a channel 262 sized to receive the spine 80 is defined between the prongs 256, 260.

In the illustrative embodiment, the prongs and body are formed as a single, monolithic component from a plastic such as polyethylene. In other embodiments, it may be formed as separate components, which may be later assembled into a single device. Some or all of the tool 250 may be formed from a metallic material such as, for example, stainless steel or a combination of metal and plastic materials.

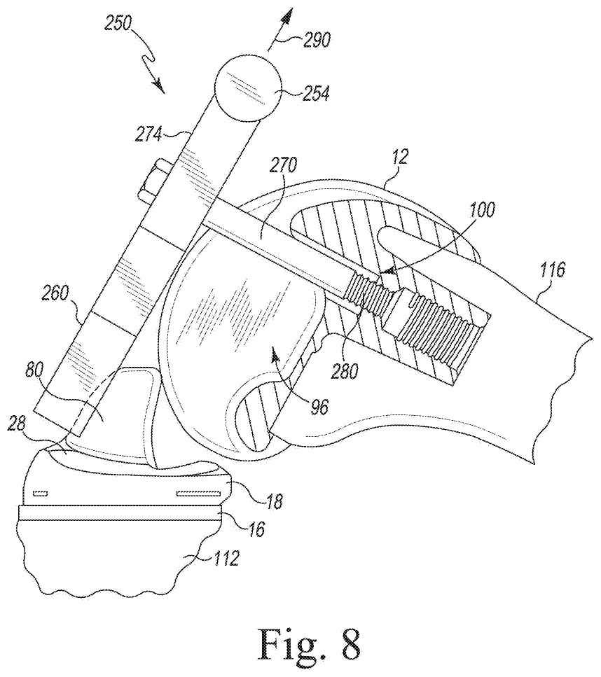

The tool 250 also includes a threaded fastener 270 that is moveably attached to the elongated body 252. As shown in FIG. 7, the fastener 270 includes a head 272 positioned on an outer side 274 of the body 252, and a shaft 276 that extends through an opening in the elongated body 252 to an outer tip 278 positioned on the inner side of the body 252. In the illustrative embodiment, the outer tip 278 of shaft 276 includes a plurality of threads 280, which are sized and shaped to engage the threaded walls of the aperture 100 of the femoral component 12, as shown in FIG. 8. The fastener 270 is illustratively formed from a metallic material such as, for example, stainless steel.

In use, the surgeon may place the femur in deep flexion, as shown in FIG. 8, with the tibial bearing 18 mounted on the tibial tray 16. The surgeon may align the channel 262 of the tool 250 with the spine 80 of the bearing 18 before advancing the prongs 260, 256 over the spine 80. The surgeon or other user may thread the fastener 270 into the threaded aperture 100 of the femoral component 12 to secure the fastener 270 (and hence the tool 250) to the femoral component 12. To displace the femur relative to the tibia, the surgeon may then lift the femur 116 (as indicated by arrow 290) and draw the femur and the tibia closer together, thereby positioning the femoral component 12 for seating on the tibial bearing 18. With the spine 80 positioned between the prongs 256, 260, rotation of the tibial bearing 18 is inhibited such that the bearing surfaces 28, 30 are aligned with the condyles 92, 94, respectively, of the femoral component 12 and the spine 80 remains aligned with the notch 96. When the condyles 92, 94 are engaged with the bearing surfaces 28, 30, the surgeon may disengage the threaded fastener 270 from the femoral component 12 and withdraw the tool 250.

Referring now to FIG. 9, another embodiment of an alignment tool (hereinafter alignment tool 350) is shown. The tool 350 is configured to displace the femur and the tibia to move the femoral component into position on the tibial bearing. The alignment tool 350 includes a main body 352 and a handle 354, which is sized to be gripped by a surgeon or other user, that extends from an upper end 356 of the body 352. At the opposite end 358 of the body, the tool 350 also includes a threaded fastener 370 that is moveably attached to the elongated body 352.

As shown in FIG. 9, the fastener 370 includes a head 372 positioned on an outer side 374 of the body 352, and a shaft 376 that extends through an opening in the elongated body 352 to an outer tip 378 positioned on the inner side of the body 352. In the illustrative embodiment, the outer tip 378 of shaft 376 includes a plurality of threads 380, which are sized and shaped to engage the threaded walls of the aperture 100 of the femoral component 12. The fastener 370, body 352, and the handle 354 are illustratively formed from a metallic material such as, for example, stainless steel.

In use, the surgeon may place the femur in deep flexion, as shown in FIG. 9, with the tibial bearing 18 mounted on the tibial tray 16. The surgeon or other user may thread the fastener 370 into the threaded aperture 100 of the femoral component 12 to secure the fastener 370 (and hence the tool 350) to the femoral component 12. To displace the femur relative to the tibia, the surgeon may lift the femur 116 and draw the femur and the tibia closer together, while holding the tibial bearing 18 in alignment with the notch 96 by hand, thereby positioning the femoral component 12 and tibial component 18 for engagement. When the condyles 92, 94 are engaged with the bearing surfaces 28, 30, the surgeon may disengage the threaded fastener 370 from the femoral component 12 and withdraw the tool 350.

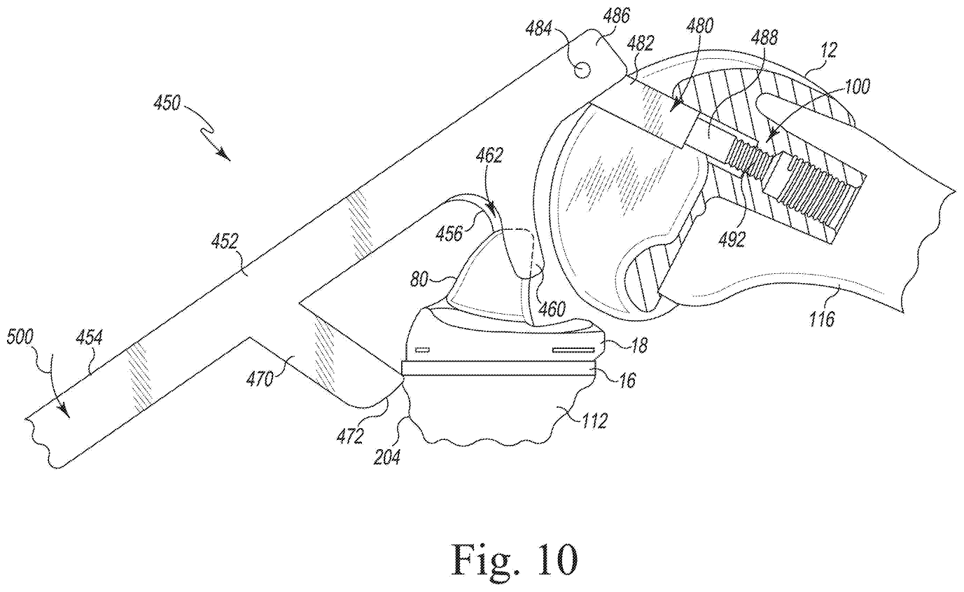

Referring now to FIG. 10, another embodiment of an alignment tool (hereinafter alignment tool 450) is shown. Like the alignment tools 150, 250, the tool 450 is configured to hold the tibial bearing in position and displace the femur and the tibia to move the femoral component into position on the tibial bearing. The alignment tool 450 includes an elongated body 452 having a handle 454, which is sized to be gripped by a surgeon or other user, at one end 458 of the body 452. A medial prong 456 extends from the elongated body 452, and the alignment tool 450 includes a lateral prong 460 also extending from the body 452. The lateral prong 460 is spaced apart from the medial prong 456 such that a channel 462 sized to receive the spine 80 is defined between the prongs 456, 460. Some or all of the tool 450 may be formed from a plastic such as, for example, polyethylene, a metallic material such as, for example, stainless steel, or a combination of metal and plastic materials.

The alignment tool 450 also includes a tab 470 extending from the elongated body 452. The tab 470 includes a convex distal surface 472 that is shaped to engage an anterior surface 204 of the patient's tibia 112. In the illustrative embodiment, the tab 470 is shaped to act against the patient's tibia, while the prongs 456, 460 maintain the tibial bearing in rotational position to seat the femoral component 12 on the tibial bearing 18.

The tool 450 also includes a threaded fastener 480 that is movably attached to the elongated body 452. As shown in FIG. 10, the fastener 480 includes a body 482 that is coupled to the tool 450 via a pin joint 484 at an upper end 486 of the body 482. The fastener 480 includes a shaft 488 that extends to an outer tip 490. In the illustrative embodiment, the outer tip 490 of shaft 488 includes a plurality of threads 492, which are sized and shaped to engage the threaded walls of the aperture 100 of the femoral component 12.

In use, the surgeon may place the femur in deep flexion, as shown in FIG. 10, with the tibial bearing 18 mounted on the tibial tray 16. The surgeon may align the channel 462 of the tool 450 with the spine 80 of the bearing 18 before advancing the prongs 460, 456 over the bearing surfaces 28, 30. The surgeon or other user may thread the fastener 480 into the threaded aperture 100 of the femoral component 12 to secure the fastener 480 (and hence the tool 450) to the femoral component 12. To displace the femur relative to the tibia, the surgeon may rotate the handle 454 of the tool 450 downward (as indicated by arrow 500) to position the tab 470 in contact with the anterior surface 204 of the tibia.

The surgeon may continue pushing down on the handle to raise the femur (or lower the tibia), while drawing the femur and the tibia closer together (by a combination of pushing backward with the tab 470 on the tibia and drawing the femur forward along the prongs 456, 460), thereby positioning the femoral component 12 for seating on the tibial bearing 18. With the spine 80 positioned between the prongs 456, 460, rotation of the tibial bearing 18 is inhibited such that the bearing surfaces 28, 30 are aligned with the condyles 92, 94, respectively, of the femoral component 12 and the spine 80 remains aligned with the notch 96. When the condyles 92, 94 are engaged with the bearing surfaces 28, 30, the surgeon may disengage the threaded fastener 480 from the femoral component 12 and withdraw the tool 450.

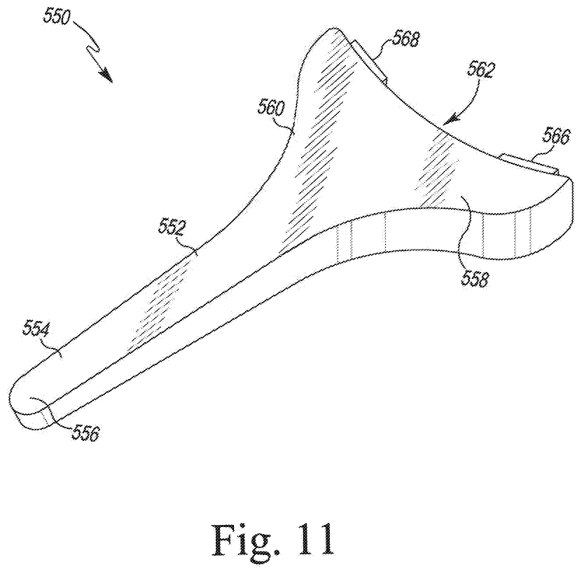

Referring now to FIG. 11, another embodiment of an alignment tool (hereinafter alignment tool 550) is shown. The tool 550 is configured to hold the tibial bearing in position while the femur and/or the tibia are displaced. The alignment tool 550 includes an elongated body 552 having a handle 554, which is sized to be gripped by a surgeon or other user, at one end 556 of the body 552. The elongated body 552 has an engagement head 558 positioned at the opposite end 560. In the illustrative embodiment, the engagement head 558 includes a concave curved surface 562 that is shaped to match the convex curved anterior surface 564 (see FIG. 1) of the tibial bearing 18. A pair of tabs 566, 568 extend from the surface 562. Each tab 566, 568 is sized to be positioned in corresponding slots 570, 572 (see FIG. 1) defined in the anterior surface 564 of the bearing 18. It should be appreciated that the engagement head 558 may be sized to extend over the anterior surface of the tibial tray 16 and the anterior surface 204 of the patient's tibia.

In use, the surgeon may place the femur in deep flexion, with the tibial bearing 18 mounted on the tibial tray 16. The surgeon may then advance the engagement head 558 into contact with the tibial bearing 18 to position the tabs 566, 568 in the slots 570, 572 of the bearing 18. The surgeon or other user may grip the handle 554 to prevent rotation of the bearing 18 while drawing the femur and tibia closer together. When the condyles 92, 94 of the femoral component 12 are engaged with the bearing surfaces 28, 30 of the tibial bearing 18, the surgeon may disengage the tabs 566, 568 from the tibial bearing 18 and withdraw the tool 550.

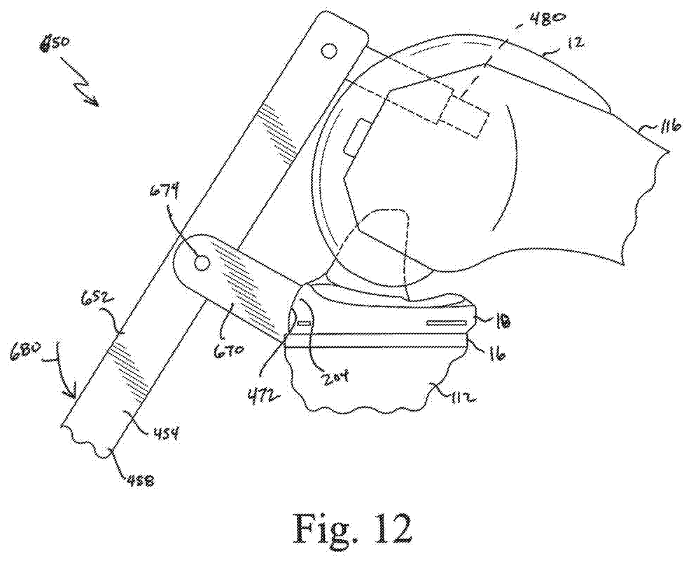

Referring now to FIGS. 12-19, variants of the embodiments of the orthopaedic surgical instruments shown in FIGS. 5-11, as well as additional embodiments of the orthopaedic surgical instrument for use with the components of the orthopaedic prosthesis system of FIG. 1 are shown.

As shown in FIG. 12, another embodiment of the alignment tool (hereinafter alignment tool 650) includes features similar to the alignment tool 450 shown and described above in regard to FIG. 10. Similar features in the tool 650 will be identified with the same reference numbers used to identify those features in the tool 450. Like the alignment tool 450, the tool 650 is configured to hold the tibial bearing in position and displace the femur and the tibia to move the femoral component into position on the tibial bearing. The alignment tool 650 includes an elongated body 652 having a handle 454, which is sized to be gripped by a surgeon or other user, at one end 458 of the body 652. A threaded fastener 480 is movably attached to the elongated body 652 in a manner similar to that shown and described above regarding the tool 450. Some or all of the tool 650 may be formed from a plastic such as, for example, polyethylene, a metallic material such as, for example, stainless steel, or a combination of metal and plastic materials.

The alignment tool 650 also includes a moveable tab 670 extending from the elongated body 652. The tab 670 includes a concave distal surface 472 that is shaped to engage an anterior surface 204 of the tibial bearing and the anterior surface of the tibial tray. In the illustrative embodiment, the tab 670 is shaped to act against those components and the patient's tibia and maintain the tibial bearing in rotational position to seat the femoral component 12 on the tibial bearing 18. In the illustrative embodiment, the tab 670 is pivotally coupled to the elongated body 652 via a pin 674, which permits the tab 670 is pivot relative to the body 652.

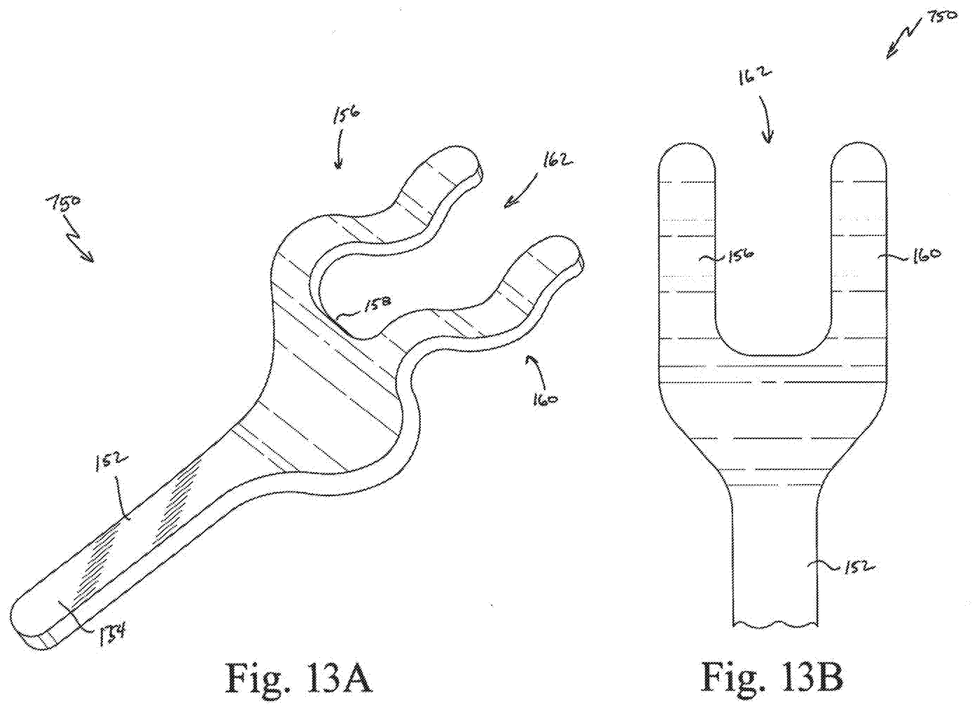

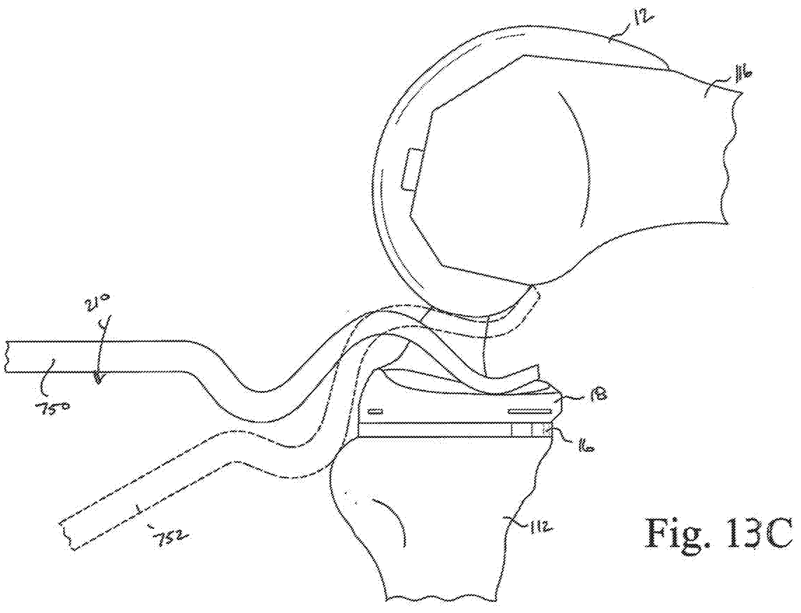

Referring now to FIGS. 13A-C, another embodiment of the alignment tool (hereinafter alignment tool 750) includes features similar to the alignment tool 150 shown and described above in regard to FIG. 5. Similar features in the tool 750 will be identified with the same reference numbers used to identify those features in the tool 150. The alignment tool 750 includes an elongated body 152 having a handle 154 sized to be gripped by a surgeon or other user. A medial prong 156 extends from an end 158 of the elongated body 152. The alignment tool 750 includes a lateral prong 160 also extending from the end 158 that is spaced apart from the medial prong 156. A channel 162 sized to receive the spine 80 is defined between the prongs 156, 160. The tool 750 is shown in multiple positions in FIG. 13C, with the second position shown in broken line and identified by the number 752.

As described above in regard to the tool 150, some or all of the tool may be formed from a metallic material such as, for example, stainless steel or a combination of metal and plastic materials. In the case of tool 750, each of the prongs 156, 160 is formed from a metallic substrate and a plastic shell that encases the metallic substrate. The elongated body 152 is formed from a metallic material that is a single, monolithic component with metallic substrates of prongs 156, 160.

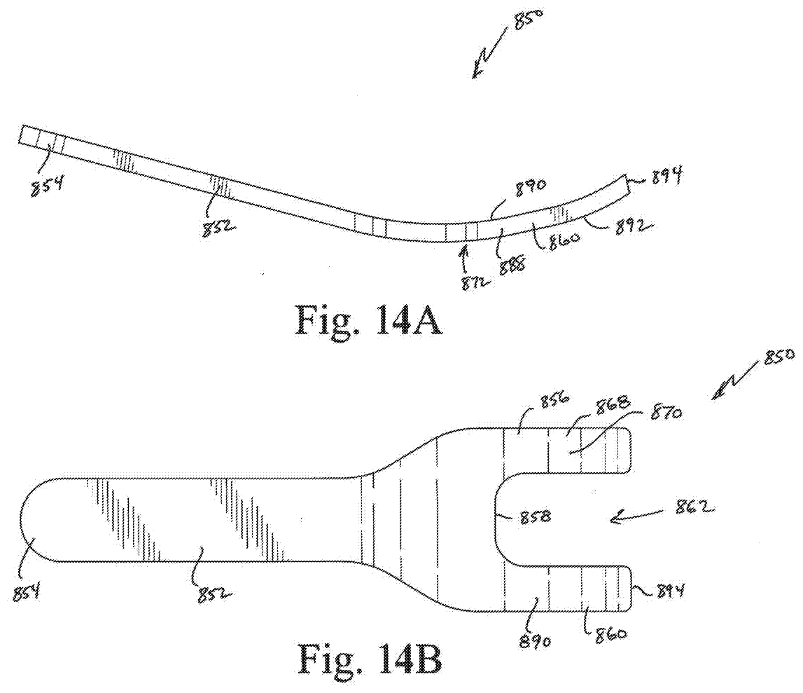

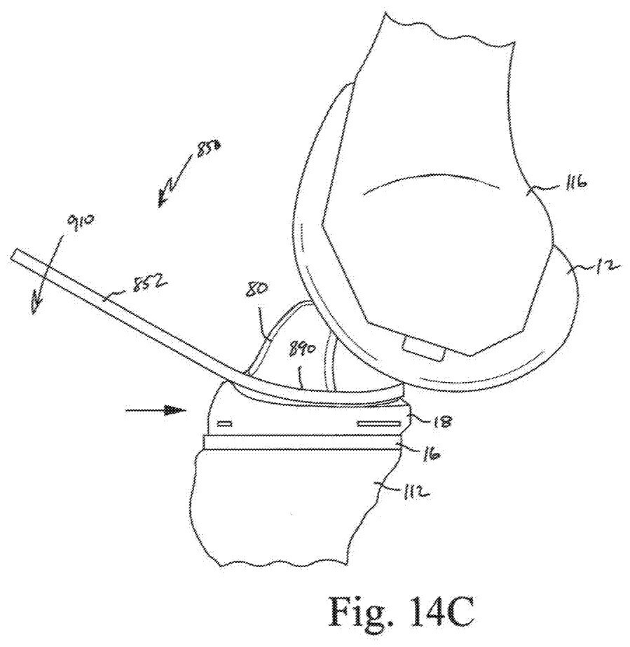

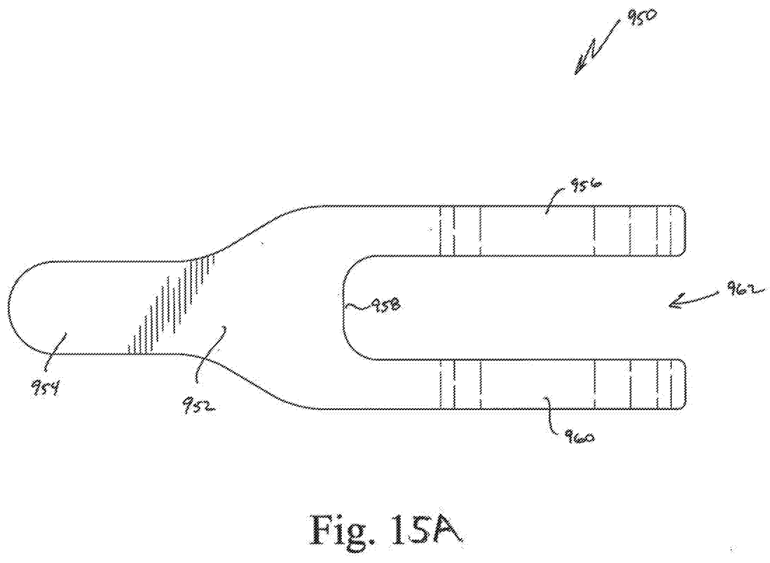

Referring now to FIGS. 14A-14C, another embodiment of the alignment tool (hereinafter alignment tool 850) is shown. The alignment tool 850 includes an elongated body 852 having a handle 854 sized to be gripped by a surgeon or other user. A medial prong 856 extends from an end 858 of the elongated body 852. The alignment tool 850 includes a lateral prong 860 also extending from the end 858 that is spaced apart from the medial prong 856. A channel 862 sized to receive the spine 80 of the tibial bearing is defined between the prongs 856, 860. In the illustrative embodiment, the tool 850 is formed as a single, monolithic component from a plastic such as polyethylene. In other embodiments, it may be formed as separate components, which may be later assembled into a single device. Some or all of the tool 850 may be formed from a metallic material such as, for example, stainless steel or a combination of metal and plastic materials.

The medial prong 856 includes an arm 868 that has a concave proximal surface 870 shaped to engage the convex condyle surface 20 of the femoral component 12. The medial prong 856 also includes a convex distal surface 872 that is positioned opposite the proximal surface 870. In the illustrative embodiment, the convex distal surface 872 is configured to engage the concave bearing surface 26 of the tibial bearing 18. The medial prong 156 also includes an outer tip defined at the end of the arm 868.

The lateral prong 860 includes an arm 888 that has a concave proximal surface 890 shaped to engage the convex condyle surface 22 of the femoral component 12. The lateral prong 860 also includes a convex distal surface 892 that is positioned opposite the proximal surface 890. In the illustrative embodiment, the convex distal surface 892 is configured to engage the concave bearing surface 28 of the tibial bearing 18. The lateral prong 160 also includes an outer tip 894 defined at the end of the arm 888.

In use, the surgeon may place the femur in deep flexion, as shown in FIG. 14C, with the tibial bearing 18 mounted on the tibial tray 16. The surgeon may align the channel 862 of the tool 850 with the spine 80 of the bearing 18 before advancing the prongs 860, 856 over the bearing surfaces 28, 30. As the prongs 860, 856 move over the bearing surfaces 28, 30, the spine 80 is received in the channel 862 and the tips of the prongs are engaged with the condyle surfaces 20, 22. To displace the femur and the tibia, the surgeon may rotate the handle 854 of the tool 850 downward (as indicated by arrow 910).

The surgeon may continue pushing down on the handle to raise the femur (or lower the tibia), while drawing the femur and the tibia closer together (by a combination of pushing backward and drawing the femur forward along the prongs 856, 860), thereby positioning the femoral component 12 for seating on the tibial bearing 18. With the spine 80 positioned between the prongs 856, 860, rotation of the tibial bearing 18 is inhibited such that the bearing surfaces 28, 30 are aligned with the condyles 92, 94, respectively, of the femoral component 12 and the spine 80 remains aligned with the notch 96. When the condyles 94, 92 are engaged with the concave proximal surfaces 870, 890 of the tool 850 over the bearing surfaces 30, 28, the surgeon may withdraw the prongs 856, 860 from between the femoral component 12 and the tibial bearing 18, thereby allowing the femoral component 12 to seat on the bearing 18.

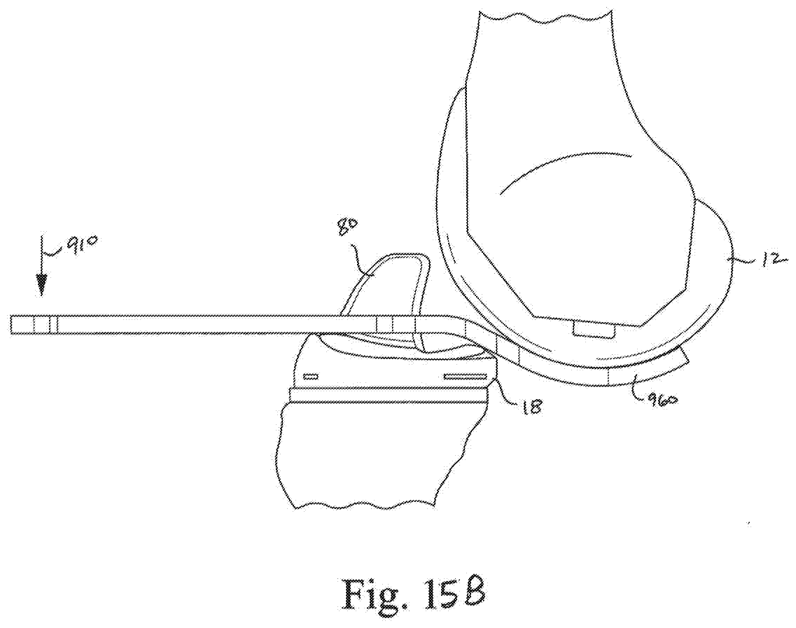

Referring now to FIGS. 15A-15B, another embodiment of the alignment tool (hereinafter alignment tool 950) is shown. The alignment tool 950 includes an elongated body 952 having a handle 954 sized to be gripped by a surgeon or other user. A medial prong 956 extends from an end 958 of the elongated body 952. The alignment tool 950 includes a lateral prong 960 also extending from the end 958 that is spaced apart from the medial prong 956. A channel 962 sized to receive the spine 80 of the tibial bearing is defined between the prongs 956, 960. In the illustrative embodiment, the tool 950 is formed as a single, monolithic component from a plastic such as polyethylene. In other embodiments, it may be formed as separate components, which may be later assembled into a single device. Some or all of the tool 950 may be formed from a metallic material such as, for example, stainless steel or a combination of metal and plastic materials.

As shown in FIG. 15A, the general configuration of tool 950 is similar to the configuration of tool 850. The channel 962 is longer or deeper than the channel 862 defined between the prongs 856, 860 of the tool 850, which permits the tool 950 to be advanced over the bearing 18 and the prongs 956, 960 positioned under the condyle surfaces 20, 22 of the femoral component 12, as shown in FIG. 15B.

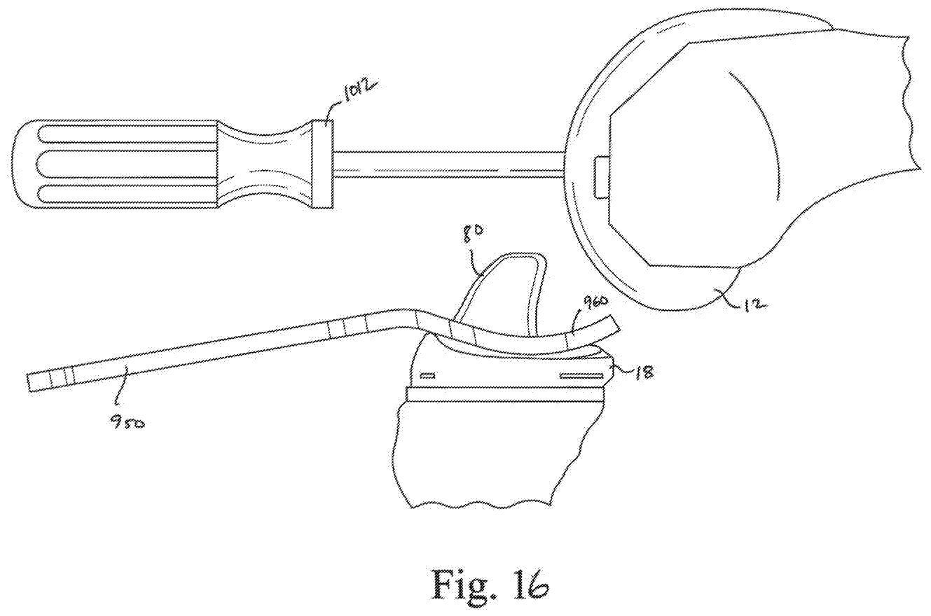

To displace the femur and the tibia, the surgeon may rotate the handle 954 of the tool 950 downward (as indicated by arrow 910), drawing the femur and the tibia closer together (by a combination of pushing backward while rotating the handle 954 downward and drawing the femur forward with the prongs 956, 960), thereby positioning the femoral component 12 for seating on the tibial bearing 18. As shown in FIG. 16, another tool 1012 may be used in conjunction with the tool 950 to draw the femur toward the tibia. The tool 1012 includes a threaded end (not shown) configured to engage the threaded walls of the aperture 100 of the femoral component 12.

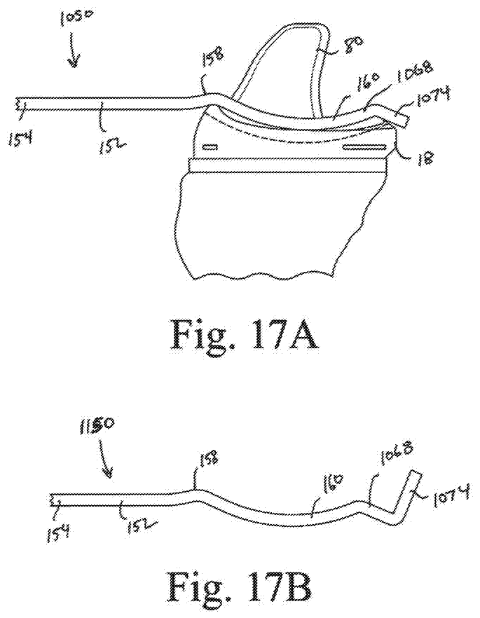

Referring now to FIGS. 17A and 17B, other embodiments of the alignment tool (hereinafter alignment tools 1050, 1150, respectively) include features similar to the alignment tool 150 shown and described above in regard to FIG. 5. Similar features in the tools 1050, 1150 will be identified with the same reference numbers used to identify those features in the tool 150. Each of the alignment tools 1050, 1150 includes an elongated body 152 having a handle 154 sized to be gripped by a surgeon or other user. A lateral prong 160 extends from an end 158 of the elongated body 152. Each of the alignment tools 1050, 1150 also includes a medial prong (not shown) extending from the end 158 that is spaced apart from the lateral prong 160. A channel (not shown) sized to receive the spine 80 is defined between the prongs.

As shown in FIGS. 17A and 17B, the lateral prong 160 of each tool includes an outer flange 1074, which extends away from the arm 1068 of the prong 160. The lateral prong 160 of each tool also includes an outer flange (not shown), which extends away from the arm of the prong 160 and has a configuration similar to the outer flange 1074 of each tool. As shown in FIGS. 17A and 17B, the outer flanges 1074 may extend in different directions to permit different ways to engage the femoral component and, in some cases, cause different forms of motion of the femur relative to the tibia. For example, the outer flanges 1074 of the tool 1050 in FIG. 17A is configured to advance below the femoral component 12 without the need to angle or displace the tibia and tibial components. The outer flanges 1074 of the tool 1150 in FIG. 17B, on the other hand, requires the surgeon to angle the tool 1150 to slide the flanges 1074 under the femoral component 12, thereby permitting the flanges 1074 to “hook” onto the femoral component 12 when the handle is rotated downward.

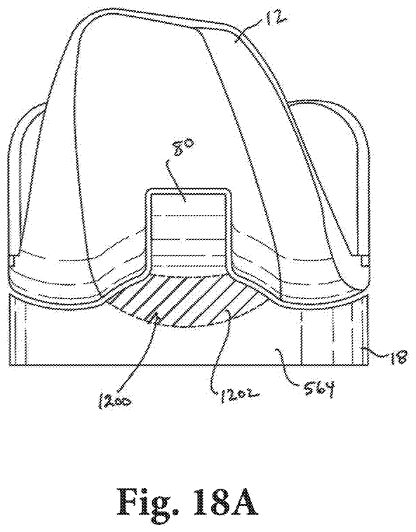

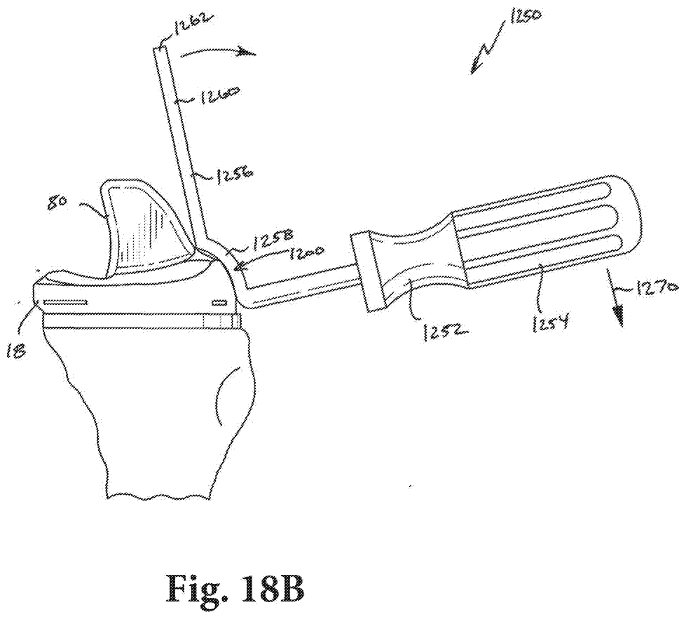

As shown in FIGS. 1 and 18A, the bearing 18 includes a divot 1200 that is defined in its anterior surface 564. In the illustrative embodiment, a concave surface 1202 extending from the base of the spine 80 defines the divot 1200. When the femoral component 12 is positioned on the bearing 18, the divot 1200 is aligned with the intercondylar notch 96 of the component 12.

Referring now to FIG. 18B, another embodiment of the alignment tool (hereinafter alignment tool 1250) is shown. The alignment tool 1250 includes an elongated body 1252 having a handle 1254 sized to be gripped by a surgeon or other user. A prong 1256 extends from the end of the body 1252 at an oblique angle relative to the handle 1254. Some or all of the tool 1250 may be formed from a metallic material such as, for example, stainless steel or a combination of metal and plastic materials.

The prong 1256 includes a curved shaft 1258 that is shaped to be received in the divot 1200 of the bearing 18 and a femoral engagement shaft 1260 that extends from the curved shaft 1258 to a proximal tip 1262. The proximal tip 1262 is shaped to engage the base 1264 (see FIG. 2) of the anterior flange 90 of the femoral component 12.

In use, the surgeon may place the femur in deep flexion, with the tibial bearing 18 mounted on the tibial tray 16. The surgeon may position the proximal tip 1262 of the alignment tool 1250 into the intercondylar notch 96 and into engagement with the base 1264. By applying a downward force in the direction indicated by arrow 1270 in FIG. 18B, the surgeon may advance the curved shaft 1258 into contact with the divot 1200. With the shaft 1258 engaged with the divot 1200, the surgeon may continue pushing down on the handle to raise the femur (or lower the tibia), while drawing the femur and the tibia closer together (by a combination of pushing backward and drawing the femur forward with the prong 1256), thereby positioning the femoral component 12 for seating on the tibial bearing 18.

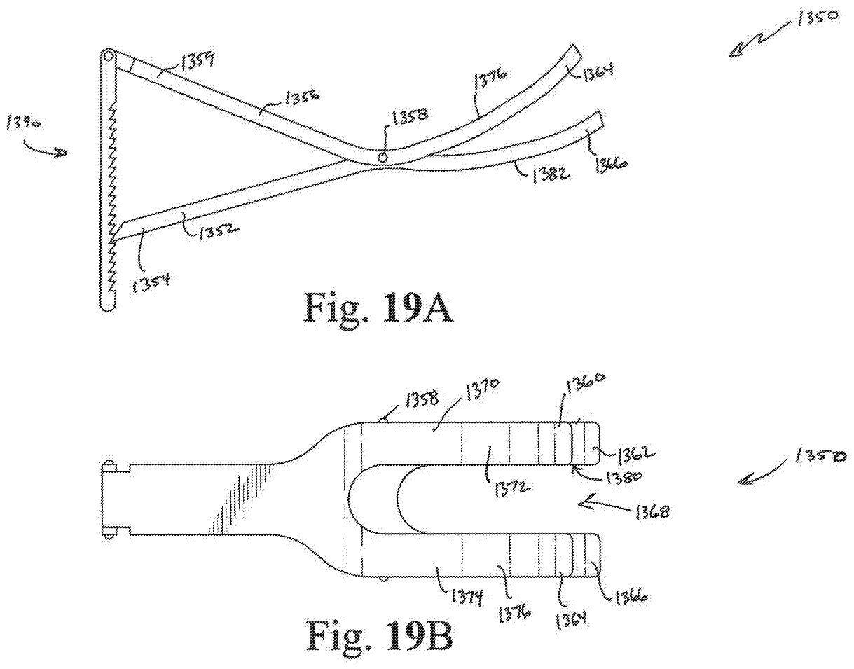

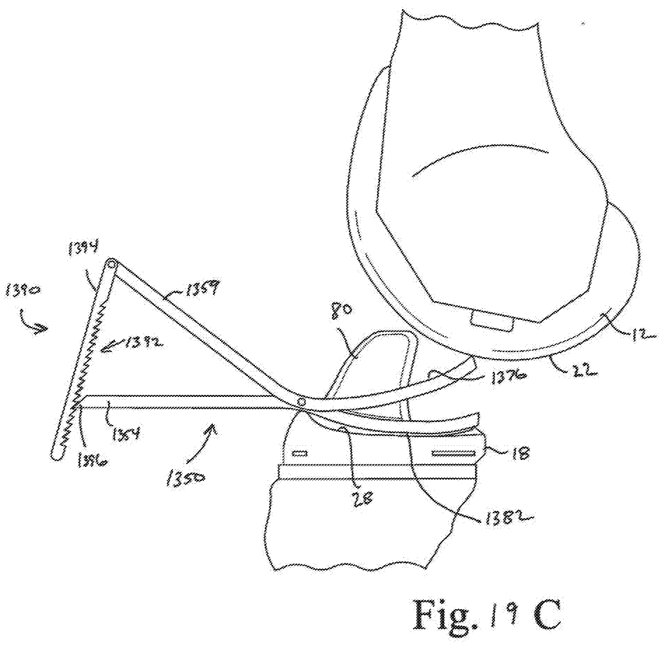

Referring now to FIGS. 19A-C, another embodiment of the alignment tool (hereinafter alignment tool 1350) is shown. The alignment tool 1350 includes a first elongated body 1352 having a handle 1354 sized to be gripped by a surgeon or other user. The alignment tool 1350 also includes a second elongated body 1356 pivotally coupled the first elongated body via a pin 1358. The second elongated body 1356 also has a handle 1359 sized to be gripped by a surgeon or other user.

Each of the bodies 1352, 1356 includes a medial prong 1362, 1360, respectively, and a lateral prong 1366, 1364, respectively, and a channel 1368 sized to receive the spine 80 that is defined between the prongs. As shown in FIG. 19B, the prongs 1360, 1362 of the body 1356 are positioned above and aligned with the prongs 1364, 1366 of the body 1352.

The medial prong 1360 of the first elongated body 1352 includes an arm 1370 that has a concave proximal surface 1372 shaped to engage the convex condyle surface 20 of the femoral component 12. The lateral prong 1366 of the first elongated body 1352 includes an arm 1374 that has a concave proximal surface 1376 shaped to engage the convex condyle surface 22 of the femoral component 12. The medial prong 1362 of the second elongated body 1356 includes a convex distal surface 1380 that is configured to engage the concave bearing surface 26 of the tibial bearing 18. The lateral prong 1366 also includes a convex distal surface 1382 that is configured to engage the concave bearing surface 28 of the tibial bearing 18.

In use, the surgeon may align the channels 1368 of the tool 1350 with the spine 80 of the bearing 18 before advancing the prongs 1360, 1362, 1364, 1366 over the bearing surfaces 28, 30. As the prongs move over the bearing surfaces 28, 30, the spine 80 is received in the channel 862 and the tips of the prongs 1360, 1362 are engaged with the condyle surfaces 20, 22, as shown in FIG. 19C. To displace the femur relative to the tibia, the surgeon may rotate the handle 1359 downward, toward the handle 1354, thereby causing prongs 1360, 1364, and hence the femur, to move upward relative to the tibia.

The surgeon may lock the handle 1358 in position relative to the handle 1356 using the locking mechanism 1390, which includes a plurality of teeth 1392 defined on a rod 1394 pivotally coupled to the handle 1358. The teeth 1392 are sized and shaped to be engaged by the end 1396 of the handle 1354, as shown in FIG. 19C, to maintain the handle 1358 in position.

Some or all of the tool 1350 may be formed from a metallic material such as, for example, stainless steel or a combination of metal and plastic materials.

Following from the above description, it should be apparent to those of ordinary skill in the art that, while the methods and apparatuses herein described constitute exemplary embodiments of the present invention, the invention contained herein is not limited to any precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

1. An orthopaedic surgical instrument, comprising:
a displacement device configured to displace the tibia and femur to permit the surgeon to position a femoral component for seating on a tibial bearing, and
a retaining mechanism configured to maintain the tibial bearing in rotational alignment with the femoral component while seating the femoral component on the tibial bearing.

2. The orthopaedic surgical instrument of claim 1, wherein the displacement device includes a pair of prongs sized and shaped to engage a pair of condyles of the femoral component.

3. The orthopaedic surgical instrument of claim 2, wherein the pair of prongs sized and shaped to engage a bearing surfaces of the tibial bearing.

4. The orthopaedic surgical instrument of claim 2, wherein the displacement device includes a tab configured to engage an anterior surface of the tibia.

5. The orthopaedic surgical instrument of claim 2, wherein each prong includes an outer flange sized to be positioned in a gap between the femoral component and the tibial bearing.

6. The orthopaedic surgical instrument of claim 2, wherein the retaining mechanism includes a channel defined between the pair of prongs, the channel being sized to receive a spine of the tibial bearing component.

7. The orthopaedic surgical instrument of claim 1, wherein the displacement device includes a fastener configured to engage the femoral component to move the femoral component relative to the tibial bearing component.

8. The orthopaedic surgical instrument of claim 7, wherein the fastener is threaded, and the femoral component includes a threaded aperture positioned at a base of an intercondylar notch.

9. The orthopaedic surgical instrument of claim 8, wherein the retaining mechanism includes a pair of prongs and a channel defined between the pair of prongs, the channel being sized to receive a spine of the tibial bearing.

10. The orthopaedic surgical instrument of claim 8, wherein the displacement device includes a tab configured to engage an anterior surface of the tibia.

11. An orthopaedic prosthesis system, comprising:
a femoral component including a pair of condyles and an intercondylar notch positioned between the pair of condyles,
a tibial tray,
a tibial bearing configured to rotatably mount to the tibial tray, the tibial bearing including a pair of proximal surfaces configured to engage the pair of condyles and a spine positioned between the pair of proximal surfaces, the spine being sized to be received in the intercondylar notch of the femoral component, and
an orthopaedic surgical instrument including (i) a displacement device configured to displace a patient's tibia and femur to permit the surgeon to position the femoral component for engagement with the tibial bearing, and (ii) a retaining mechanism configured to maintain the tibial bearing in rotational alignment with a femoral component while the femoral component is being positioned for engagement with the tibial bearing.

12. The orthopaedic prosthesis system of claim 11, wherein the displacement device includes a pair of prongs sized and shaped to engage the pair of condyles of the femoral component and the pair of bearing surfaces of the tibial bearing.

13. The orthopaedic prosthesis system of claim 12, wherein the retaining mechanism includes a channel defined between the pair of prongs, the channel being sized to receive a spine of the tibial bearing.

14. The orthopaedic prosthesis system of claim 12, wherein each prong of the pair of prongs includes a concave upper surface shaped to engage a condyle of the pair of condyles.

15. The orthopaedic surgical instrument of claim 11, wherein the displacement device includes a fastener configured to engage the femoral component to move the femoral component relative to the tibial bearing.

16. The orthopaedic surgical instrument of claim 15, wherein the fastener is threaded, and the femoral component includes a threaded aperture positioned at a base of an intercondylar notch.

17. The orthopaedic surgical instrument of claim 11, wherein retaining mechanism includes an engagement head configured to confront at least one of the tibial bearing, a tibial tray, and an anterior surface of a patient's tibia.

18. The orthopaedic surgical instrument of claim 17, wherein the engagement head includes a pair of tabs sized to be positioned in a pair of slots of the tibial bearing.

19. The orthopaedic surgical instrument of claim 11, wherein the retaining mechanism includes a pair of prongs and a channel defined between the pair of prongs, the channel being sized to receive a spine of the tibial bearing.
